(19) United States
(12) Patent Application Publication
(10) Pub. No.: US 2022/0409247 A1
(43) Pub. Date: Dec. 29, 2022

(54) Fixing Plate with Locking Mechanism Assembly

(71) Applicant: Tyber Medical LLC, Bethlehem, PA (US)

(72) Inventors: Jeffrey Tyber, Breinigsville, PA (US); David Hannah, Nazareth, PA (US); Michal Zentko, Flanders, NJ (US); Brad Juchno, Yardley, PA (US)

(73) Assignee: Tyber Medical LLC, Bethlehem, PA (US)

(21) Appl. No.: 17/628,365

(22) PCT Filed: Jul. 16, 2020
(86) PCT No.: PCT/US20/42227

§ 371 Date: Jan. 19, 2022

Related U.S. Application Data
(60) Provisional application No. 62/885,863, filed on Aug. 13, 2019.
(60) Provisional application No. 62/876,593, filed on Jul. 19, 2019.

(51) Int. Cl. A61B 17/70; A61B 17/80

(52) U.S. Cl. CPC A61B 17/7059; A61B 17/8042; A61B 17/808

(57) ABSTRACT

A retaining plate with locking mechanism assembly includes a retaining plate having a first screw hole extending therethrough and a spring blocker rotatably mounted in plate proximate to the first screw hole. The spring blocker includes a body portion, a first land extending radially from the body portion above the retaining plate, and a first wing extending tangentially from the first land in a first arc. When a first screw is inserted into the first screw hole, when the spring blocker is rotated to a first position, the first land extends over a portion of the first screw. When the spring blocker is rotated to a second position, the first wing extends over the portion of the first screw. When the spring blocker is rotated to a third position, neither the first land nor the first wing extends over the portion of the first screw.

### BACKGROUND OF THE INVENTION

### Field of the Invention

The present invention relates to a fixing plate that is used to fixedly attach multiple bones or bone pieces together.

### Description of the Related Art

Medical implants are often used to secure adjacent bones or bone fragments together. One potential problem, however, with the screws used to secure an implant in a patient is that, over time, it is possible for the screws to work their way out of the bone, potentially causing pain to the patient and requiring a clinician to either remove or re-insert the screws, which can cause additional pain to the patient and possibly open the patient to risk of infection.

It would be beneficial to provide a fixation assembly that minimizes or eliminates the risk of screws backing out.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a retaining plate with locking mechanism assembly that includes a retaining plate having a first screw hole extending therethrough and a spring blocker rotatably mounted in plate proximate to the first screw hole. The spring blocker includes a body portion, a first land extending radially from the body portion above the retaining plate, and a first wing extending tangentially from the first land in a first arc. When a first screw is inserted into the first screw hole, when the spring blocker is rotated to a first position, the first land extends over a portion of the first screw. When the spring blocker is rotated to a second position, the first wing extends over the portion of the first screw. When the spring blocker is rotated to a third position, neither the first land nor the first wing extends over the portion of the first screw.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a plate assembly according to a first exemplary embodiment of the present invention attached to a spine;

FIG. 2 is a top plan view of a locking mechanism used with the plate of FIG. 1;

FIG. 3 is a perspective view of the locking mechanism of FIG. 2;

FIG. 4 is a top plan view of with the plate of FIG. 1;

FIG. 5 is a perspective view of the plate of FIG. 1;

FIG. 6 is a sectional view of the plate of FIG. 1, taken along lines 6-6 of FIG. 4;

FIG. 7 is a top plan view of an alternative plate assembly according to the present invention;

FIG. 8 is a perspective view of the plate assembly of FIG. 7;

FIG. 8A is a perspective view of an expandable retaining plate according to an exemplary embodiment of the present invention;

FIG. 9 is a top plan view of a one-piece spring blocker for use with the plate assembly of FIG. 1 or FIG. 7;

FIG. 9A is a perspective view of the spring blocker of FIG. 9;

FIG. 10 is a top plan view of the one piece spring blocker of FIG. 9 used with the plate assembly of FIG. 1;

FIG. 11 is a top plan view of a spring blocker relative to screws to be blocked, with the spring blocker in a static insertion position;

FIG. 12 is a top plan view of the spring blocker and screws of FIG. 11, with the blocker in a closed and locking position;

FIG. 13 is a top plan view of the spring blocker and screws of FIG. 11, with the blocker in an open position;

FIG. 14 is a perspective view of the spring blocker of FIGS. 11-13 inserted into a plate;

FIG. 15 is a perspective view of a spring blocker inserted into a Z-plate;

FIG. 15A is a side elevational view of the spring blocker and Z-plate of FIG. 15, with an interbody fusion device;

FIG. 16 is perspective view of an inserter attached to an implant with the Z-plate with spring blocker of FIG. 15 inserted there between;

FIG. 17 is an enlarged side elevational view of the inserter attached to the implant with the Z-plate with spring blocker of FIG. 16;

FIG. 18 is a top plan view of a spring blocker for blocking a single screw in a plate according to an alternative exemplary embodiment of the present invention, with the spring blocker blocking the screw;

FIG. 19 is a top plan view of the spring blocker with screw and plate of FIG. 18, with the spring blocker rotated to locate the wing of the spring blocker over the screw;

FIG. 20 is a top plan view of the spring blocker with screw and plate of FIG. 18, with the spring blocker rotated to allow the screw to be removed from the plate;

FIG. 21 is a top plan view of a spring blocker according to an alternative embodiment of the present invention;

FIG. 22 is a top plan view of a spring blocker according to another alternative embodiment of the present invention;

FIG. 23 is a sectional view of the spring blocker of FIG. 22, taken along lines 23-23 of FIG. 22;

FIG. 24 is a top plan view of a spring blocker according to another alternative embodiment of the present invention, with an optional cam lock in an unlocked position; and

FIG. 25 is a top plan view of the spring blocker of FIG. 24, with the cam lock in a locked position.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

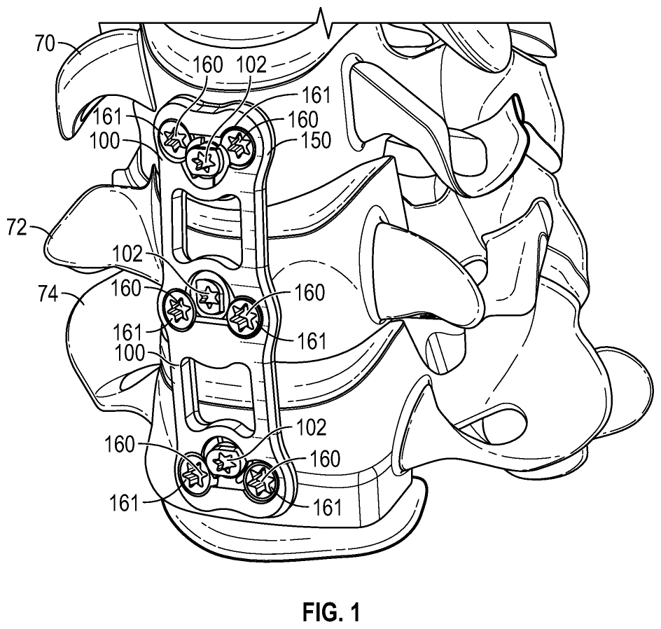

Referring to the Figures, the present invention provides a cervical plating platform with enhanced locking features to prevent implanted screws from backing out after implantation. In an exemplary embodiment, shown in FIGS. 1-6, a locking spring blocker assembly 100 is used to secure adjacent screws 160 in a retaining plate 150. As shown in FIG. 1, retaining plate 150 is used as a spinal plate.

Retaining plate 150 has a plurality of fixation portions, each fixation portion having a pair of adjacent screw holes 161 formed there in for receiving fixation screws 160.

Spring blocker assembly 100 incorporates a singular locking mechanism with dual functionality. The locking mechanism includes a spring blocker 102, with a locking cam 104 inserted coaxially over spring blocker 102. Spring blocker 102 can be constructed from Nitinol, titanium, stainless steel, or other suitable biocompatible material. Spring blocker 102 is arranged proximate to the pair of screw holes 161 such that spring blocker 102 acts as a retaining device to retain screws 160 in screw holes 161 after screws 160 are inserted into bone.

Spring blocker 102 includes a generally circular central body portion 106 having a through opening 108 formed therein. Through opening 108 is sized and shaped to accept locking cam 104.

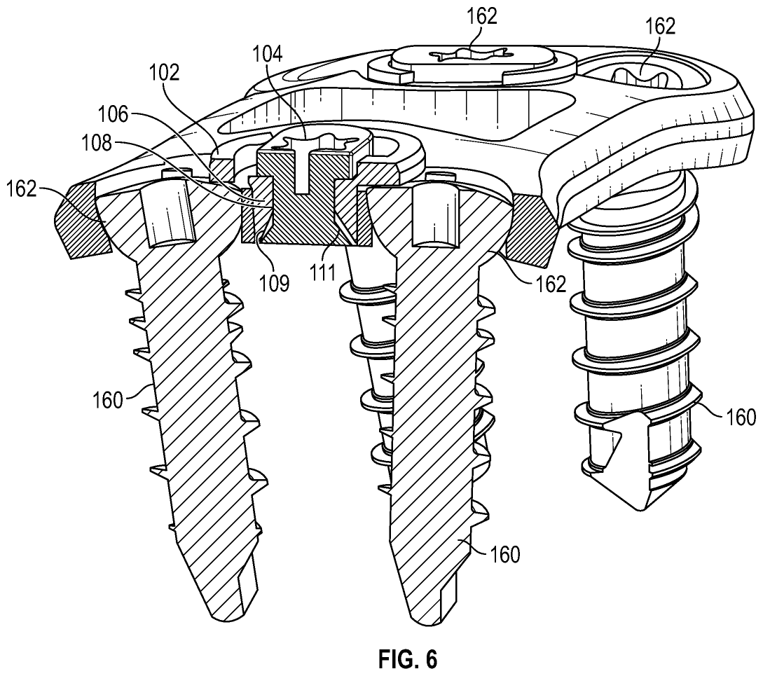

Through opening 108 extends downwardly through central body portion 106 and flares outwardly into a frustoconical shaped lower recess 109 to engage a similarly shaped lip 111 in locking cam 104, as shown in FIG. 6, yet allow body portion 106 and locking cam 104 to rotate freely in retaining plate 150.

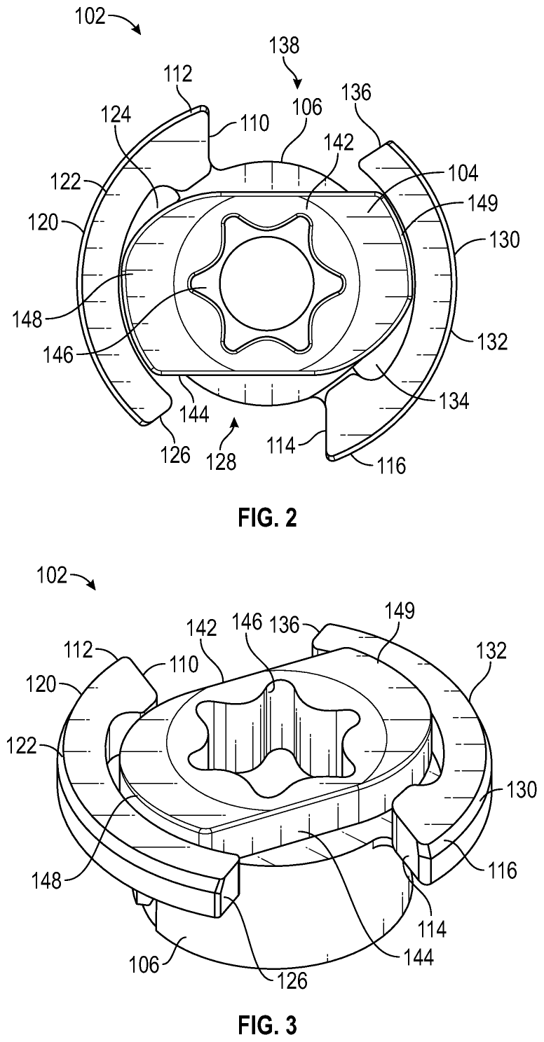

Referring specifically to FIGS. 2 and 3, a first land 110 extends radially outwardly from body portion 106 and extends over an arc of about 10 degrees. First land 110 has a beveled arcuate first outer edge 112 with a radius of curvature. Similarly, a second land 114 extends outwardly from body portion 106 diametrically across body portion 106 from first land 110. Second land 114 has a beveled second arcuate outer edge 116 with the same radius of curvature as first outer edge 112.

A beveled first wing 120 extends tangentially from first land 110 in an arc such that an outer edge 122 of first wing 120 has the same radius of curvature as first arcuate edge 112. First wing 120 extends about 90 degrees around the perimeter of body portion 106. A gap 124 is provided between first wing 120 and body portion 106 so that a free end 126 of first wing 120 can flex relative to first land 110.

Similarly, a beveled second wing 130 extends tangentially from second land 114 in an arc such that an outer edge 132 of second wing 130 has the same radius of curvature as second arcuate edge 116. Second wing 130 extends about 90 degrees around the perimeter of body portion 106 in the same direction as first wing 120. A gap 134 is provided between second wing 130 and body portion 106 so that a free end 136 of second wing 130 can flex relative to second land 114.

A first space 128 is provided between free end 126 of first wing 120 and second land 114. Similarly, a second space 138 is provided between free end 136 of second wing 130 and first land 110. Spaces 128, 138 are sized to allow the heads of screws 160 to be uncovered by spring blocker assembly 100 for removal of screws 160, if desired.

Locking cam 104 includes a generally oblong shaped body 140 with parallel longitudinal sides 142, 144 on either side of a recess 146. In an exemplary embodiment, recess 146 can be for a Torx® head driver, although those skilled in the art will recognize that recess 146 can be shaped to accept other types of drivers.

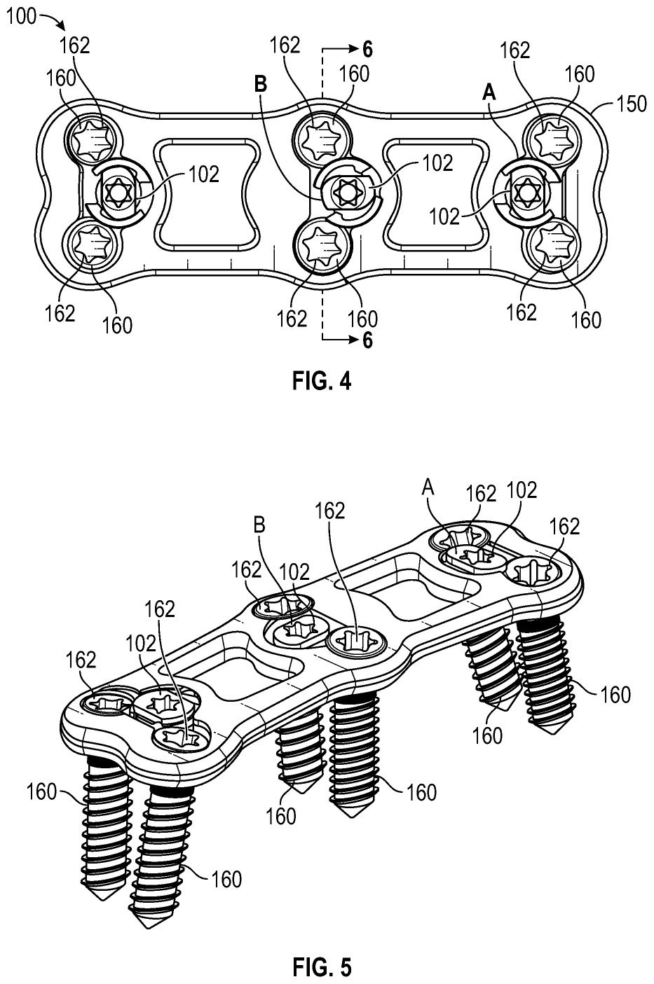

Sides 142, 144 are sufficiently long such that, when locking cam 104 is rotated to the position shown at “A” in FIGS. 4 and 5, lateral lobes 148, 149 extend over the heads 162 of screws 160. When locking cam 104 is rotated to the position shown at “B” in FIGS. 4 and 5, lateral lobes 148, 149 do not extend over heads 162 of screws 160, so that screws 160 can be removed, if desired. Wings 120, 130 extend radially outwardly of lateral lobes 148, 149 in any rotational position of locking cam 104 relative to spring blocker 102.

Spring blocker assembly 100 can be used in a one-step locking process or a two-step locking process. In a one-step locking process, retainer plate 150 is aligned with three adjacent vertebrae 70, 72, 74, as shown in FIG. 1. Screws 160 are inserted into their respective vertebrae 70, 72, 74. As screws 160 are inserted, screw head 162 engages beveled wings 120, 130, and biases wings 120, 130 inward, toward the center of spring blocker assembly 100. After screws 160 are inserted far enough into the vertebrae such that heads 162 are below wings 120, 130, wings 120, 130 snap back into their unbiased positions to cover a portion of screw head 162, preventing screw head 162 from backing out of the respective vertebra.

In a two-step locking process, in addition to the process described immediately above, locking cam 140 is rotated from position “B” in FIG. 4 to position “A” in FIGS. 4 and 5 so that lateral lobes 148, 149 extend over the heads 162 of screws 160, adding another level of securement of screws 160 in their respective vertebra 70, 72, 74. Those skilled in the art will recognize that the second step (rotating locking cam 140) is an optional step and can be omitted if desired. As shown in FIG. 4, spring blocker 102 does not extend co-linearly with the screw holes that retain screws 160.

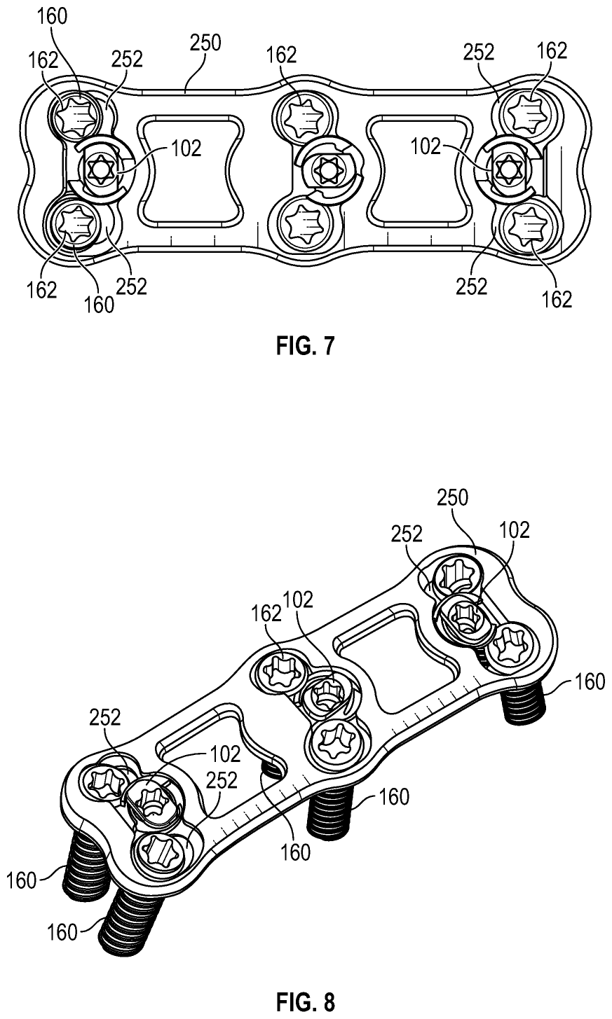

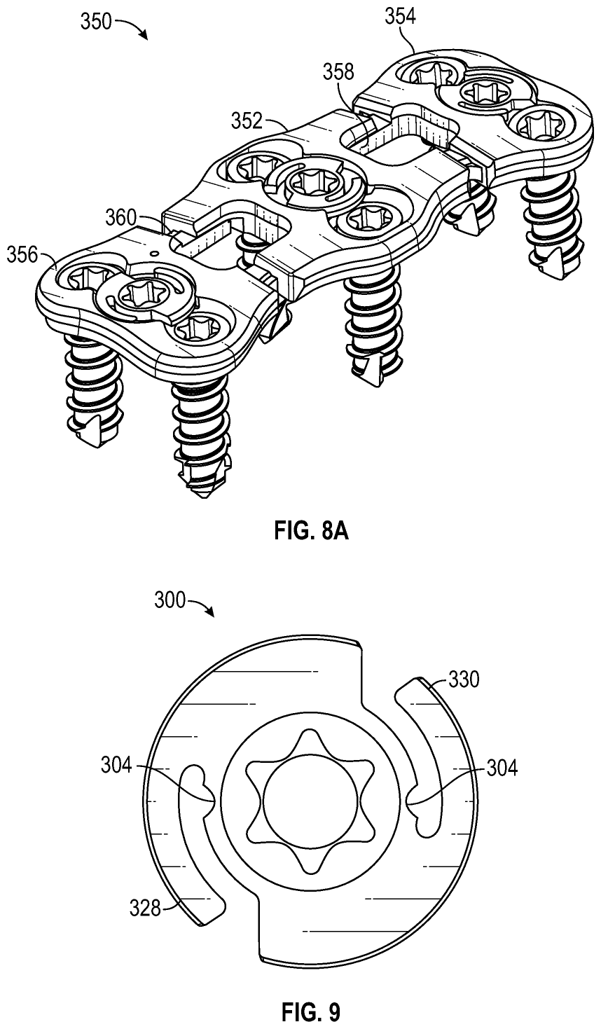

Referring to FIGS. 7 and 8, a retaining plate 250 can be used instead of retaining plate 150. Retaining plate 250 can include oversized slots 252 on either end of plate 250 for screws 160 so that screws 160 can shift their location in holes 252 as the patient's spine settles and compresses over time.

Referring to FIG. 8A, an expandable retaining plate 350 is shown. Plate 350 includes a center portion 352 and opposing end portions 354, 356 that are slidingly attached to center portion 352 by rails 358, 360, respectively. Rails 358, 360 allow end portions 354, 356 to move relative to central portion 352 to accommodate for spine settling over time.

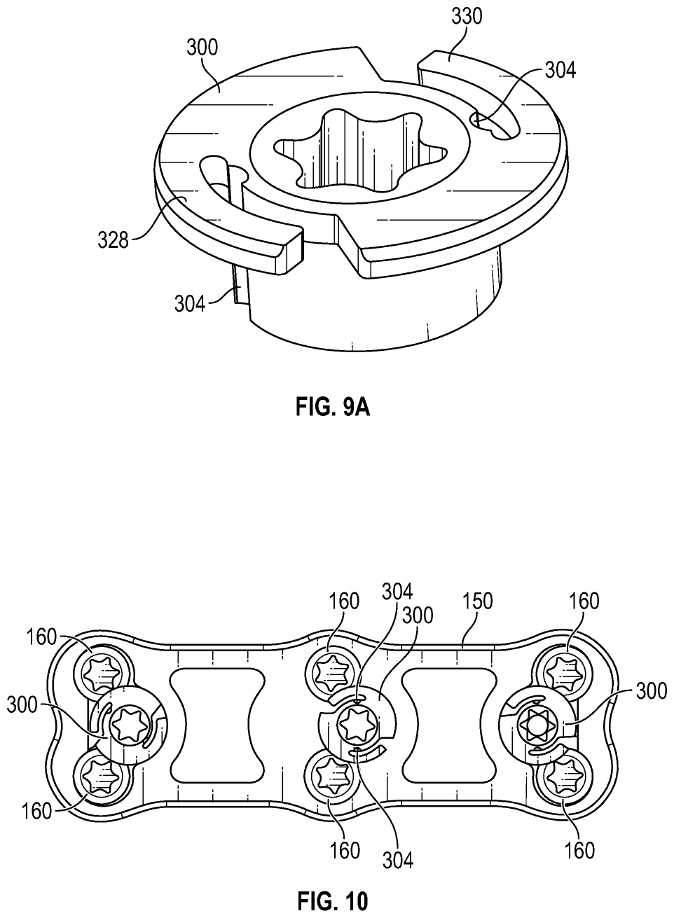

Referring to FIGS. 9, 9A, and 10, a one-piece spring blocker 300 can be used to secure screws 160 in a retaining plate 150, or a retaining plate 250. Spring blocker 300 is a one-piece device and can be constructed from Nitinol, titanium, stainless steel, or other suitable biocompatible material.

Spring blocker 300 is similar to spring blocker 102, except that spring blocker 300 includes a pair of diametrically opposing detents 304 that extend from a top 306 of spring blocker 300, along a body 308 of spring blocker 300, to a bottom 310 of spring blocker 300. Detents 304 each engage a protrusion (not shown) in plate 150, 250 to resist rotation of spring blocker 300 in either a clockwise or a counter-clockwise direction. Detents 304 and their associated protrusions are located relative to each other to engage when wings 328, 330 are over the heads 162 of screws 160, restricting the ability of screws 160 to work themselves out of the bone.

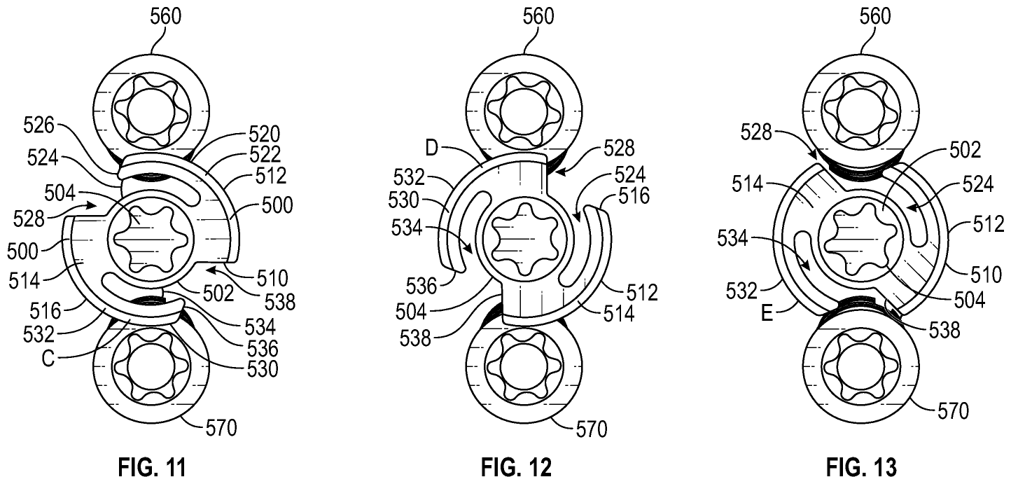

FIGS. 11-13 show an alternative embodiment of a locking spring blocker assembly 500 according to an exemplary embodiment of the present invention. Locking spring blocker assembly 500 includes a generally circular central body portion 502 having a blind opening 504 formed therein. Blind opening 504 is sized and shaped to accept a driver (not shown). Alternatively, opening 504 can be a through opening.

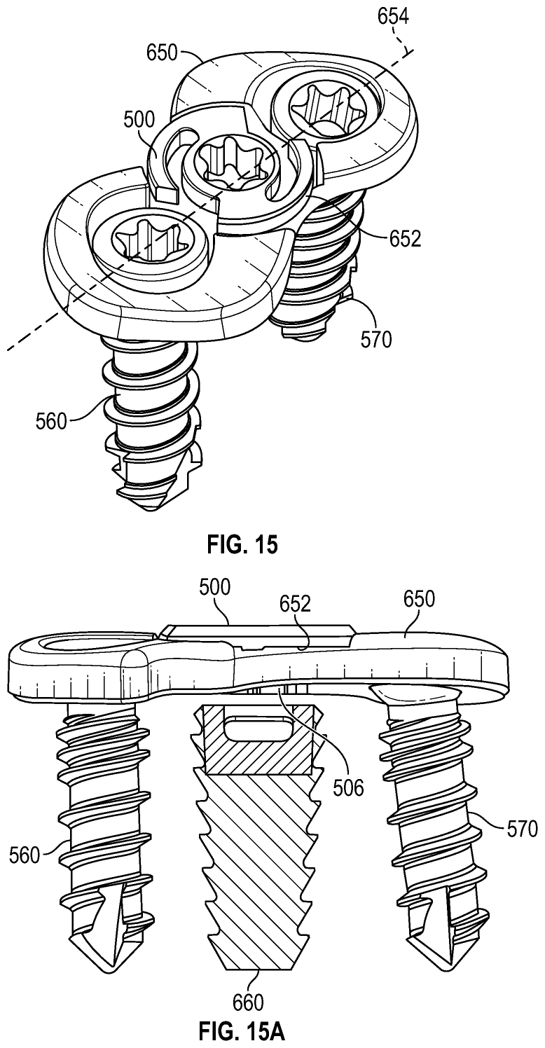

Central body portion 502 extends downwardly from blind opening 504 and flares outwardly into a frustoconical shaped lower portion 506 (shown in FIG. 15A) to engage a recess in a retaining plate, yet allow body portion 502 to rotate freely within the recess, as shown in FIG. 10.

A first land 510 extends radially outwardly from body portion 502 and extends over an arc of about 45 degrees. First land 510 has a beveled arcuate first outer edge 512 with a radius of curvature. Similarly, a second land 514 extends outwardly from body portion 502 diametrically across body portion 502 from first land 510. Second land 514 has a beveled second arcuate outer edge 516 with the same radius of curvature as first outer edge 512.

A beveled first wing 520 extends tangentially from first land 510 in an arc such that an outer edge 522 of first wing 520 has the same radius of curvature as first arcuate edge 512. First wing 520 extends about 90 degrees around the perimeter of body portion 502. A gap 524 is provided between first wing 520 and body portion 502 so that a free end 526 of first wing 520 can flex relative to first land 510.

Similarly, a beveled second wing 530 extends tangentially from second land 514 in an arc such that an outer edge 532 of second wing 530 has the same radius of curvature as second arcuate edge 516. Second wing 530 extends about 90 degrees around the perimeter of body portion 502 in the same direction as first wing 520. A gap 534 is provided between second wing 530 and body portion 502 so that a free end 536 of second wing 530 can flex relative to second land 514.

A first space 528 is provided between free end 526 of first wing 520 and second land 514. Similarly, a second space 538 is provided between free end 536 of second wing 530 and first land 510. Spaces 528, 538 are sized to allow the heads of screws 560, 570 to be uncovered by spring blocker assembly 500, as shown in FIG. 13, for removal of screws 560, 570, if desired.

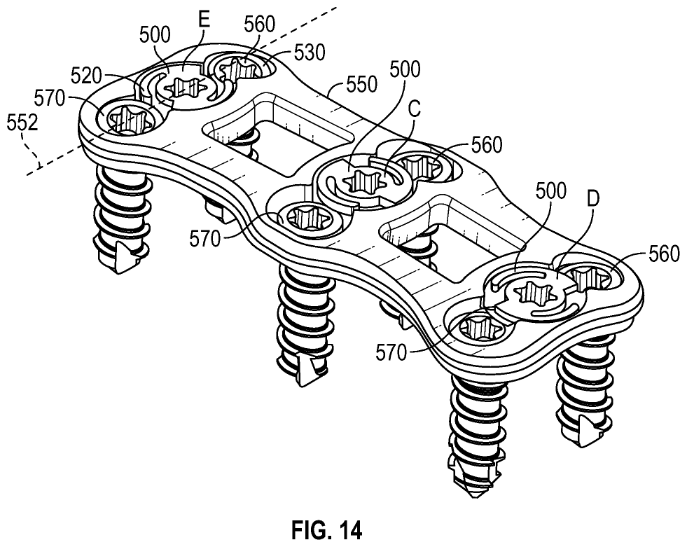

Referring to FIG. 14, a plate 550 is provided with spring blocker assembly 500 rotatably attached to plate 550, with a pair of retaining screws 560, 570 provided on diametrically opposed sides of spring blocker assembly 500. Screws 560, 570 are located relative to spring blocker assembly 500 such that when spring blocker assembly 500 is rotated to the position “C” shown in FIG. 11, as screws 560, 570 are threaded into bone (not shown) through plate 550, the beveled surfaces of wings 520, 530 can snap over the heads of screws 560, 570. To lock screws 560, 570 in place, spring blocker assembly 500 can be rotated to the position “D” shown in FIG. 12 to securely lock screws 560, 570 into pace and prevent screws 560, 570 from inadvertently backing out. To remove screws 560, 570, spring blocker assembly 500 can be rotated to the position “E” shown in FIG. 13 so that the heads of screws 560, 570 are aligned with spaces 528, 538, respectively, allowing screws 560, 570 to be unthreaded from the bone and removed from plate 550. As shown in FIG. 14, screw hole with screw 560, spring blocker assembly 500, and screw hole with screw 570 extend co-linearly. Each of positions C, D, and E are reflected in FIG. 14.

FIG. 15 shows spring blocker assembly 500 inserted into a Z-plate 650. A flat surface 652 is formed in the top of Z-plate 650 to accommodate spring blocker assembly 500 so that spring blocker assembly 500 does not extend vertically above the height of Z-plate 650.

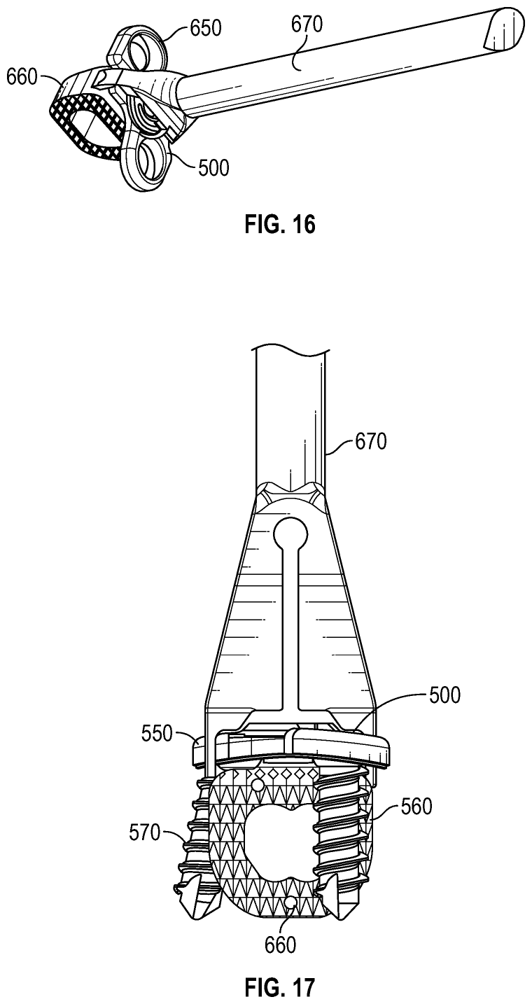

FIGS. 16 and 17 show an inserter 670 with spring blocker assembly 500, Z-plate 650, and implant 660. Implant 660 can be a spinal implant to be inserted between adjacent vertebrae. Alternatively, implant 660 can be a wedge implant for an osteotomy. In an exemplary embodiment, inserter 670 is attached to implant 660 along side walls of implant 660, while Z-plate 650 with spring blocker assembly 500, although restrained by inserter 670 and implant 660, are not attached to inserter 670 and are free to “float” between inserter 670 and implant 660.

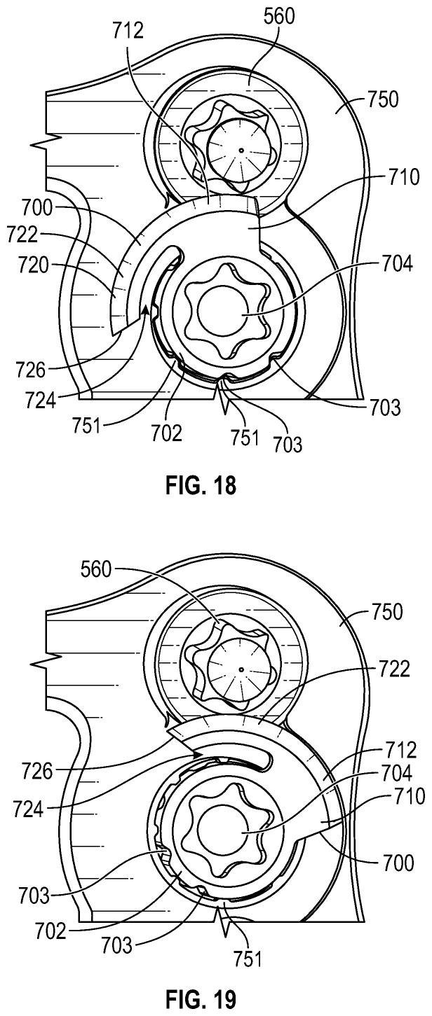

FIGS. 18-20 show top plan views of a locking spring blocker assembly 700 according to an alternative exemplary embodiment of the present invention. While spring blocker assembly 500 is used with two screws 560, 570 in a Z-plate 650, spring blocker assembly 700 can be used with only a single screw 560 in a plate 750 or in a single blocking condition for plate 650. Consequently, spring blocker assembly 700 has some aspects that are similar/identical to spring blocker assembly 500, but is also omitting several aspects.

Similar to spring blocker assembly 500, spring blocker assembly 700 has a generally circular central body portion 702 having a blind opening 704 (or, alternatively, a through opening) formed therein. Blind opening 704 is sized and shaped to accept a driver (not shown).

Central body portion 702 extends downwardly from blind opening 704 and flares outwardly into a frustoconical shaped lower portion 706 to engage a recess in a retaining plate, yet allow body portion 702 to rotate freely within the recess, as shown in FIG. 10.

Body portion 702 includes a plurality of detents 703 formed around an exterior perimeter of body portion 702. Detents 703 are engaged by protrusions 751 in plate 750 to add an additional locking feature to restrict unwanted rotation of spring blocker assembly 700 with respect to plate 750.

A first land 710 extends radially outwardly from body portion 702 and extends over an arc of about 45 degrees. First land 710 has an arcuate first outer edge 712 with a radius of curvature. Unlike spring blocker assembly 500, spring blocker assembly 700 has no second land.

A first wing 720 extends tangentially from first land 710 in an arc such that an outer edge 722 of first wing 720 has the same radius of curvature as first arcuate edge 712. First wing 720 extends about 90 degrees around the perimeter of body portion 702. A gap 724 is provided between first wing 720 and body portion 702 so that a free end 726 of first wing 720 can flex relative to first land 710. Unlike spring blocker assembly 500, spring blocker assembly 700 has no second wing.

A first space 728 is provided between free end 726 of first wing 720 and second land 714. Unlike spring blocker assembly 500, spring blocker assembly 700 has no second space.

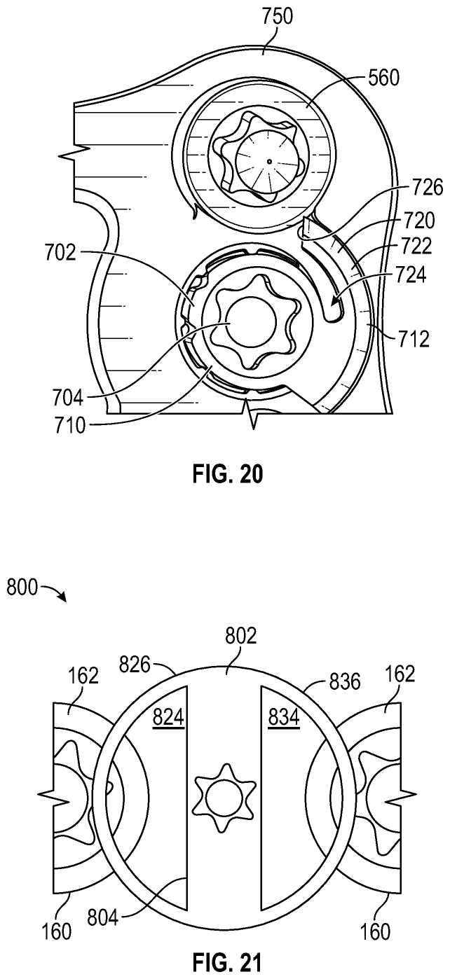

When the spring blocker 700 is rotated to a first position, as shown in FIG. 18, first land 710 extends over a portion of the screw 560. When the spring blocker 700 is rotated to a second position, as shown in FIG. 19, first wing 720 extends over the portion of the screw 560. When spring blocker 700 is rotated to a third position, as shown in FIG. 20, neither first land 710 nor first wing 720 extend over the portion of the screw 560.

Referring now to FIG. 21, a spring blocker assembly 800 is shown. Spring blocker assembly 800 is similar to spring blocker assembly 500 except that, instead of wings 520, 530 with free ends 526, 536, respectively, spring blocker assembly 800 includes a body 802 with beveled wings 826, 836 having both ends connected to body 802, such that generally “D-shaped” openings 824, 834 are formed between body 802 and each respective wing 826, 836.

As described above with respect to other embodiments, as screw 160 are inserted, screw head 162 engages beveled wings 826, 836, and biases wings 826, 836 inward, toward the center 804 of spring blocker assembly 800. After screws 160 are inserted far enough into the vertebrae such that heads 162 are below wings 826, 836, wings 826, 836 snap back into their unbiased positions to cover a portion of screw head 162, preventing screw head 162 from backing out of the respective vertebra.

While spring blocker assembly 800 is shown without a locking cam, those skilled in the art will recognize that a locking cam can be added to provide additional locking capability.

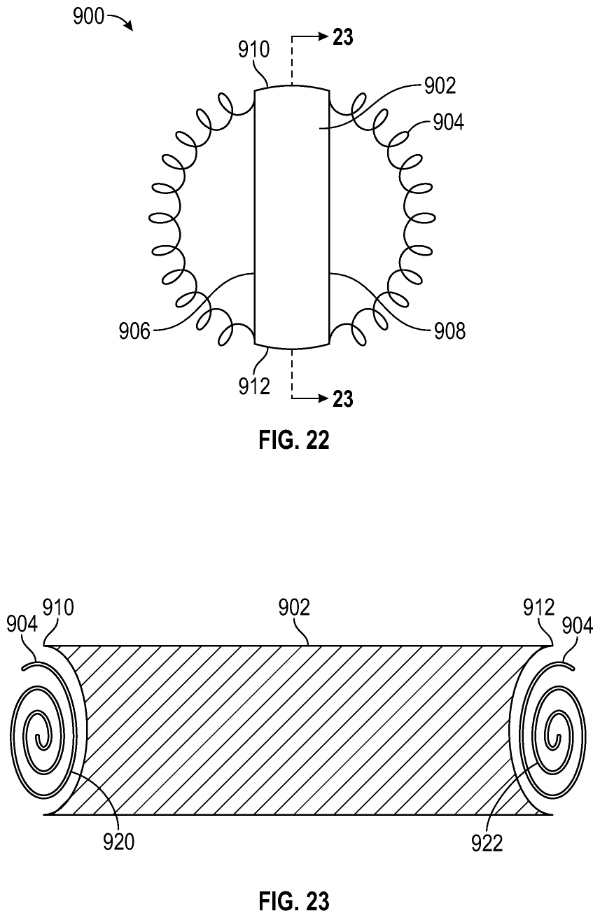

Referring now to FIG. 22, a spring blocker assembly 900 is show. Spring blocker assembly 900 includes a body 902 and a circular spring 904 that extends around body 902. Body 902 is elongate with generally parallel longitudinal sides 906, 908, with arcuate sides 910, 912 connecting longitudinal sides 906, 908 at either end. Referring to FIG. 23, arcuate sides 910, 912 have concave cross sections that form “pockets” 920, 922 sized to accept and retain spring 904.

Spring 904 is a continuous helical spring that naturally takes the form of a circle. When spring 904 is inserted into pockets 920, 922, spring still retains a circular configuration. As screws 160 are screwed into a retaining plate 150, 250, 550, 650, 750 utilizing spring blocker assembly 900, screw heads 162 force spring 904 inwardly, toward a center of spring blocker assembly 900. After screw heads 162 are below the level of spring 904, spring 904 naturally returns to its circular configuration, such that spring 904 extends over screw heads 162, retaining screw heads 162 in place.

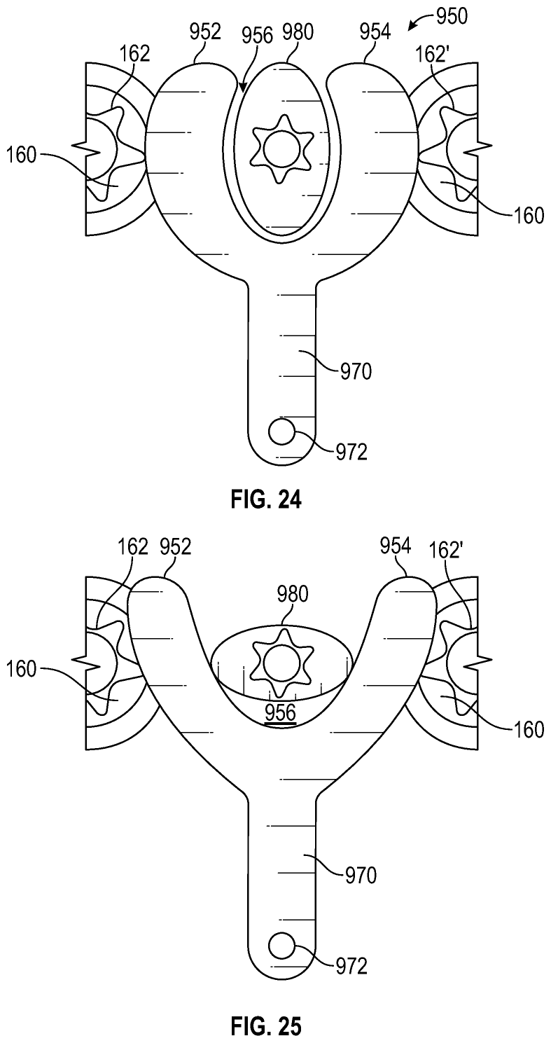

Referring to FIG. 24, a generally “Y-shaped blocker assembly 950 is shown. Blocker assembly 950 includes a first arm 952 that is sized to fit over part of a first screw head 162 and a second arm 954 that is sized to fit over part of an adjacent screw head 162′. A gap 956 between arms 952, 954 is provided to allow each arm 952, 954 to bias into gap 956 as a screw 160 is being inserted into a plate. After screw heads 162 are below the level of arm 952, 954, arm 952, 954 naturally returns to its original configuration, such that arm 952, 956 extends over screw heads 162, retaining screw heads 162 in place.

A stem 970 of blocker assembly 950 extends away from gap 956 and incudes a screw 972 that is used to secure blocker assembly 950 to a plate, such as plate 150, 250, 550, 650, 750, or any other type of plate.

Optionally, a cam lock 980 can be located in gap 956 and rotated from an unlocked position, as shown in FIG. 24, to a locked position, as shown in FIG. 25, to bias arms 952, 954 apart from each other, and further over screw heads 162, 162′ to further secure screw heads 162, 162′ into the plate.

In an exemplary embodiment, and of the components described above can be constructed from nitinol, titanium, a titanium alloy, stainless steel, or other suitable biocompatible material.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A retaining plate with locking mechanism assembly comprising:
a retaining plate having a first screw hole extending therethrough; and
a spring blocker rotatably mounted in plate proximate to the first screw hole, the spring blocker comprising:
a body portion;
a first land extending radially from the body portion above the retaining plate; and
a first wing extending tangentially from the first land in a first arc,
wherein, when a first screw is inserted into the first screw hole:
when the spring blocker is rotated to a first position, the first land extends over a portion of the first screw;
when the spring blocker is rotated to a second position, the first wing extends over the portion of the first screw; and
when the spring blocker is rotated to a third position, neither the first land nor the first wing extends over the portion of the first screw.

2. The retaining plate with locking mechanism assembly according to claim 1, wherein the body portion comprises at least one detent formed therein.

3. The retaining plate with locking mechanism assembly according to claim 1, wherein the first land comprises a beveled edge.

4. The retaining plate with locking mechanism assembly according to claim 3, wherein the first wing comprises a beveled edge.

5. The retaining plate with locking mechanism assembly according to claim 1, wherein the retaining plate further comprises a second screw hole proximate to the first screw hole, and wherein the spring blocker further comprises:
a second land extending radially from the body portion above the retaining plate, the second land being located diametrically across the body portion from the first land; and
a second wing extending tangentially from the second land in a second arc,
wherein, when a second screw is inserted into the second screw hole:
when the spring blocker is rotated to the first position, the second land extends over a portion of the second screw;
when the spring blocker is rotated to the second position, the second wing extends over the portion of the second screw; and
when the spring blocker is rotated to the third position,
neither the second land nor the second wing extend over the portion of the second screw.

6. The retaining plate with locking mechanism assembly according to claim 5, wherein the first screw hole, the spring blocker, and the second screw hole extend co-linearly.

7. The retaining plate with locking mechanism assembly according to claim 5, wherein the first screw hole, the spring blocker, and the second screw hole do not extend co-linearly.

8. The retaining plate with locking mechanism assembly according to claim 1, wherein the spring blocker further comprises a locking cam coaxially mounted on the body portion, the locking cam comprising a pair of opposing lateral lobes.

9. The retaining plate with locking mechanism assembly according to claim 8, wherein the locking cam is rotatable to a first position such that neither of the lateral lobes extends over the portion of the first screw and a second position such that one of the lateral lobes extends over the portion of the first screw.

10. The retaining plate with locking mechanism assembly according to claim 8, wherein the first arm extends radially outwardly of the lateral lobes.

11. The retaining plate with locking mechanism assembly according to claim 8, wherein the locking cam is rotatable relative to the spring blocker.

12. The retaining plate with locking mechanism assembly according to claim 1, wherein the first arm is compressible toward the body.

13. The retaining plate with locking mechanism assembly according to claim 1, wherein the retaining plate comprises a spinal plate.

14. The retaining plate with locking mechanism assembly according to claim 1, wherein the first screw hole comprises an oversized slot.

15. The retaining plate with locking mechanism assembly according to claim 1, wherein the first land extends over an arc of about 45 degrees and wherein the retaining plate is expandable.
